(19) United States
(12) Patent Application Publication
(10) Pub. No.: US 2008/0305069 A1
(43) Pub. Date: Dec. 11, 2008

(54) Cosmetic Use of Tensing Agents to Improve the Thickness of the Skin and/or the Radiance of the Complexion

(75) Inventors: Guillaume Cassin, Villebon Sur Yvette (FR); Philippe Catroux, Guitres (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/094,279

(22) PCT Filed: Nov. 17, 2006
(86) PCT No.: PCT/EP06/68633

§ 371 Date: Aug. 21, 2008

(30) Foreign Application Priority Data
Nov. 21, 2005 (FR) 0553523

(51) Int. Cl. A61K 31/765; A61K 33/24; A61K 33/12; A61P 17/00; A61K 33/06; A61K 31/74

(52) U.S. Cl. 424/78.18; 424/617; 424/724; 424/684; 424/78.31

(57) ABSTRACT

The invention relates in particular to the cosmetic use of a composition containing, in a physiologically acceptable medium, an effective amount of at least one mosaic-effect tensing agent for improving the thickness of the skin and/or promoting the radiance of the complexion. The invention also relates to the cosmetic use, in a composition containing a physiologically acceptable medium, of an effective amount of at least one mosaic-effect tensing agent as an agent intended to improve the firmness and/or elasticity and/or tonicity of the skin. The invention likewise relates to a cosmetic method of treatment of dull and/or poorly defined complexion that comprises the topical application of a composition comprising at least one mosaic-effect tensing agent to people who have a dull and/or poorly defined complexion. Mosaic-effect agents of this kind are selected in particular from (i) mineral tensing agents and (ii) tensing polymers.

The present invention relates to the field of skincare and is directed in particular to improving the appearance of the skin and/or the complexion.

The invention relates in particular to the cosmetic use of a composition containing, in a physiologically acceptable medium, an effective amount of at least one mosaic-effect tensing agent for improving the thickness of the skin and/or promoting the radiance of the complexion.

The invention also relates to the cosmetic use, in a composition containing a physiologically acceptable medium, of an effective amount of at least one mosaic-effect tensing agent as an agent intended to improve the firmness and/or elasticity and/or tonicity of the skin.

The invention likewise relates to a cosmetic method of treatment of dull and/or poorly defined complexion that comprises the topical application of a composition comprising at least one mosaic-effect tensing agent to people who have a dull and/or poorly defined complexion.

Mosaic-effect agents of this kind are selected in particular from (i) mineral tensing agents and (ii) tensing polymers.

The invention is directed in particular to the treatment of the skin of the face and/or neck. However, the compositions of the invention can also be applied to areas of the body exhibiting a loss of elasticity and/or firmness, such as the stomach and the thighs.

The skin constitutes a physical barrier between the body and its environment. It is composed of two tissues: the epidermis and the dermis.

The epidermis is a keratinizing multi-layered epithelium which undergoes continual renewal. Keratinocytes make up the primary epidermal cell population and are responsible for maintaining the epithelial structure and its barrier function. The epidermis rests on an acellular basal membrane, called the dermoepidermal junction, which ensures cohesion with the dermis.

The epidermis is composed of a number of strata of cells, the deepest of which is the basal stratum, which is composed of undifferentiated cells. Over time these cells will undergo differentiation and will migrate towards the surface of the epidermis, thereby making up the different epidermal strata, until, at the surface of the epidermis, they will form the corneocytes, which are dead cells which are removed by desquamation. This surface loss is compensated by the migration of cells from the basal stratum towards the surface of the epidermis. The process is one of continuous renewal of the skin.

The dermis is an elastic and compressible conjunctive support tissue of mesodermal origin and consists primarily of fibroblasts and an extracellular matrix which is composed of fibrous proteins (collagens and elastin) and non-fibrous proteins (proteoglycans and glycoproteins). The dermis is a feeder tissue for the epidermis, but also plays a fundamental part in the development and growth of the epidermis, and also in its differentiation. The fibroblasts and the extracellular matrix also influence the mechanical properties of the skin, particularly its elasticity, tonicity and firmness. The fibroblasts and the extracellular matrix also influence the density of the skin.

The homeostasis of the skin, and in particular of the epidermis, results from a finely regulated balance between the processes of proliferation and differentiation of the cells of the skin. These proliferation and differentiation processes are perfectly regulated: they participate in the renewal and/or regeneration of the skin and lead to the maintenance of a constant skin thickness, and in particular a constant epidermal thickness. This homeostasis of the skin is also involved in maintaining the mechanical properties of the skin.

However, this homeostasis of the skin can be affected by certain physiological factors (age, menopause, hormones, etc.) or environmental factors (UV stress, pollution, oxidizing stress, irritant stress, etc.). The regenerative potential of the epidermis becomes less great: the cells of the basal layer divide less actively, which leads in particular to a slowdown and/or decrease in epidermal renewal. Consequently, cellular renewal no longer compensates for the loss of the cells removed at the surface, leading to atrophy of the epidermis and/or to a decrease in the thickness of the skin and/or a loss of elasticity and/or tonicity and/or firmness of the skin.

These clinical signs are visible on the skin of the face and/or neck, but also on the skin of the body, particularly in areas which exhibit a loss of firmness and/or elasticity, such as the stomach and/or thighs.

The alterations in epidermal homeostasis are also manifested in a dull and/or poorly defined appearance to the complexion of the skin.

This phenomenon may be accentuated by the menopause: women complain of their skin tightening and becoming dry, or even of the appearance of xerosis. The hormonal deficits associated with the menopause are accompanied in particular by a drop in metabolic activity, which can result in a decrease in the proliferation of the keratinocytes and in an increase in epidermal differentiation.

The need is understood, therefore, to have agents capable of promoting the homeostasis of the skin in order to maintain and/or increase the thickness of the skin, particularly the skin of the face and/or neck, and thus to maintain and/or improve the mechanical properties of the skin, particularly the skin of the face and/or neck, and/or to promote radiance in the complexion.

The prior art discloses the use of soluble cosmetic agents for promoting cellular renewal. They include, for example, retinoic acid derivatives, and especially retinol, also known as vitamin A, and esterified derivatives of retinol, which have the effect of promoting proliferation of the keratinocytes and of inhibiting their differentiation, thereby making it possible to stimulate epidermal renewal, to increase the thickness of the epidermis and/or to promote radiance in the complexion.

The soluble cosmetic agents act classically via a bond to a receptor which initiates intracellular responses, leading to a regulation of the expression of proteins which are involved in the processes of epidermal proliferation and/or differentiation. This is termed a direct ‘biological’ effect.

The Applicant has now shown, surprisingly and unexpectedly, that an improvement in the homeostasis of the skin and/or in the radiance of the complexion can be obtained via a biomechanical effect provided by the topical application of an effective amount of cosmetic agents, especially mosaic-effect tensing agents.

The Applicant has shown in effect that the topical application of an effective amount of tensing agents, such as acrylic copolymers, to a model of reconstructed skin had the effect of modulating the expression of proteins involved in the homeostasis of the skin.

By ‘biomechanical effect’ according to the invention is meant the capacity of a cosmetic agent, and in particular of a tensing agent, to induce a biological response in the cells of the epidermis and/or dermis, via a mechanical effect which is effective at the surface of the skin (stratum corneum).

By ‘mechanical effect which is effective at the surface of the skin’ is meant the capacity of a cosmetic agent to induce biologically effective mechanical tensions, i.e. mechanical tensions capable of transmitting a mechanical perturbation from cell to cell or via the extracellular matrix, and involving the activation of mechanoreceptors which are present on the membranes of said cells. These cells are referred to as ‘biologically sensitive to mechanical tensions’: interest attaches in particular to the cells of the epidermis and dermis, and especially to the keratinocytes and fibroblasts.

These mechanical tensions, in contrast to conventional stimulation by soluble molecules of the kind used hitherto, have the effect of modifying, via membrane receptors or ‘mechanoreceptors’, an equilibrium which is established between the extracellular matrix and a cell, or between two adjacent cells.

The mechanical tensions are transmitted in the cell in the form of biochemical signals via membrane receptors or mechanoreceptors.

These mechanoreceptors are membrane receptors which are sensitive to mechanical tensions, in other words membrane receptors capable of inducing an intracellular biological response in response to a mechanical perturbation. They include the integrins (Pommerenke et al., Eur J Cell Biol 1996 June; 70(2): 157-64), PECAM1 receptors (Fujiwara et al., Cell struct funct 2001 February; 26(1): 11-17) or else PDGF growth factor receptors (Li et al., Cell Signal 2000 July; 12(7): 435-45).

The tensions, by inducing mechanical perturbation of these receptors, in a first step trigger activation of multiple second messengers. The tensions activate, in particular, protein tyrosine kinase (PTK), protein kinase C (PKC), the G proteins rac and cdc42, or induce the release of calcium flows. The activation of these various signalling pathways leads to the activation of protein kinases from a single family, the MAPkinases, Erk1, Erk2 and p38. The MAPKs, once activated, induce the activation of specific transcriptional factors which regulate the expression of numerous genes involved in the homeostasis of keratinocytes. These activation mechanisms are, moreover, well regulated: in the course of tensions, in particular, Erk induces the expression of MAPK phosphatases, which are known to inhibit Erk. This process allows the cells to control the signals induced by the tensions, and to prevent pathological hyperproliferation of the keratinocytes.

To the knowledge of the Applicant there has never to date been any description or suggestion of improving epidermal homeostasis and in particular the thickness of the skin and/or the radiance of the complexion by the topical application to the skin of an effective amount of a cosmetic agent having a biomechanical effect, in particular a tensing agent.

The prior art has disclosed the use of tensing agents to obtain a superficial and immediate visual effect of smoothing of the microrelief of the skin, particularly smoothing of wrinkles and fine lines. These agents are described as being capable of forming a film which causes the retraction of the stratum corneum, which is the surface, horny layer of the epidermis. The cosmetic or dermatological use of such polymer systems for attenuating the alterations in the microrelief of the skin that are associated with age is described in patent application WO 98/29091. Other tensing agents consist of dispersions of inorganic colloidal particles, particularly silica, as described in patent applications FR-A-2 823 113, FR-2 843 024 and FR-2 659 551 or in patents U.S. Pat. No. 3,819,825 and U.S. Pat. No. 4,777,041, for example. Further tensing agents are mixed silicates such as those described in patent application FR-2 816 315.

To the knowledge of the Applicant, however, there has never to date been any description of a biological effect of the tensors, and in particular of their capacity to regulate the expression of proteins involved in the homeostasis of the skin, or of their use to promote the homeostasis of the skin and in particular the thickness of the skin and/or to improve the radiance of the complexion and/or to improve the mechanical properties of the skin, in particular at the level of the face and/or neck, but also at the level of certain areas of the body (e.g. stomach, thighs).

The invention therefore in particular provides for the cosmetic use of a composition comprising, in a physiologically acceptable medium, at least one mosaic-effect tensing agent for increasing the thickness of the skin.

It likewise provides for the cosmetic use of a composition comprising, in a physiologically acceptable medium, at least one mosaic-effect tensing agent for increasing the thickness of the epidermis.

The invention provides additionally for the cosmetic use of a composition comprising, in a physiologically acceptable medium, at least one mosaic-effect tensing agent for promoting the radiance of the complexion.

The invention provides additionally for the cosmetic use of a composition comprising, in a physiologically acceptable medium, at least one mosaic-effect tensing agent for improving the density of the skin.

The invention provides additionally for the cosmetic use of a composition comprising, in a physiologically acceptable medium, at least one mosaic-effect tensing agent for promoting the regeneration and/or reorganization of the papillary dermis.

The invention likewise provides for the cosmetic use, in a composition for topical application to the skin, containing a physiologically acceptable medium, of an effective amount of at least one mosaic-effect tensing agent as an agent intended to promote the homeostasis of the skin.

In particular, the effective amount of at least one mosaic-effect tensing agent is intended to promote the homeostasis of the epidermis.

By “effective amount of tensing agent” which can be used in accordance with the invention is meant, in particular, an amount sufficient to obtain the required biological effect of the tensors in the context of the present invention, namely, in particular, an effect on the regulation of the expression of genes involved in the homeostasis of the skin.

This effective amount or effective dose can be evaluated, for example, by a DNA array method as described in the illustrative examples below, the general principle of which is as follows:
different doses of tensing agents are applied to cells in culture or to a model of epidermis and/or of reconstructed skin;
the mRNAs are extracted from said treated or untreated (control) cells and a “reverse” transcription is carried out using, for example, oligo dT and a P33-labelled deoxynucleotide triphosphate, to give labelled target cDNA sequences;
these target cDNA sequences are hybridized on dedicated minichips containing DNAs specific for the markers that are involved in the physiology of cells of the skin, and in particular in the homeostasis of the skin (called “cDNA probes”);
after washing, the amount of labelled target sequences is measured, and is compared with the control in order to evaluate the variation of expression of target genes that is induced by the topical application of said tensing agent relative to the control;
subsequently a selection is made of the effective doses or amounts for which a variation is obtained in the expression of genes involved in the proliferation (increase) and/or differentiation (decrease) in the cells of the skin relative to an (untreated) control. Advantageously, a selection is made of the effective doses for which a decrease is obtained in the expression of genes involved in the differentiation of keratinocytes (e.g. corneodesmosin, loricrin, suprabasin) and/or an increase in the genes involved in regeneration of the skin (e.g. cytokeratins) relative to a control, preferably a variation in the expression by a factor of 2 or more relative to the control.

The amount of tensing agent present in the cosmetic compositions according to the invention ranges from 0.1% to 30% by weight of active ingredient relative to the total weight of the composition. Preference will be given to using an effective amount ranging from 1% to 30% by weight of active ingredient relative to the total weight of the composition, preferably from 2% to 30% by weight, in particular from 3% to 20% by weight, preferably from 4% to 20% by weight of active ingredient relative to the total weight of the composition, and in particular an effective amount of between 6% and 10% by weight of active ingredient relative to the total weight of the composition.

According to one particular embodiment use will be made of an effective amount of tensing agent of 3% to 20% by weight of active ingredient relative to the total weight of the composition, preferably of 3% to 7% by weight of active ingredient relative to the total weight of the composition.

By “active ingredient”, the intention is to exclude the medium in which the tensing agent is optionally solubilized or in dispersion in its commercial form, as for example in the case of dispersions of colloidal particles.

The invention likewise provides for the cosmetic use, in a composition for topical application to the skin, containing a physiologically acceptable medium, of an effective amount of at least one tensing agent as an agent intended to prevent and/or reduce the alteration in the homeostasis of the skin that is induced by environmental stresses.

By “environmental stresses” are meant, in particular, in accordance with the invention, UV radiation, pollution, oxidizing stress or irritant stress.

The invention likewise provides for the cosmetic use, in a composition for topical application to the skin, containing a physiologically acceptable medium, of an effective amount of at least one mosaic-effect tensing agent as an agent intended to increase the thickness of the skin, in particular the thickness of the epidermis.

The invention likewise provides for the cosmetic use, in a composition for topical application to the skin, containing a physiologically acceptable medium, of an effective amount of at least one mosaic-effect tensing agent as an agent intended to promote the radiance of the complexion and/or to improve its luminosity.

The invention likewise provides for the cosmetic use, in a composition for topical application to the skin, containing a physiologically acceptable medium, of an effective amount of at least one mosaic-effect tensing agent as an agent intended to promote and/or improve the mechanical properties of the skin, in particular to promote and/or improve the elasticity and/or firmness and/or tonicity of the skin.

The invention likewise provides for the cosmetic use, in a composition for topical application to the skin, containing a physiologically acceptable medium, of an effective amount of at least one mosaic-effect tensing agent as an agent intended to improve the density of the skin.

The invention likewise provides for the cosmetic use, in a composition for topical application to the skin, containing a physiologically acceptable medium, of an effective amount of at least one mosaic-effect tensing agent as an agent intended to promote the regeneration and/or reorganization of the papillary dermis.

The invention likewise provides for the cosmetic use, in a composition for topical application to the skin, containing a physiologically acceptable medium, of an effective amount of at least one mosaic-effect tensing agent as an agent intended to promote the regeneration and/or reorganization of the extracellular matrix.

The invention likewise provides for the cosmetic use, in a composition for topical application to the skin, containing a physiologically acceptable medium, of an effective amount of at least one mosaic-effect tensing agent as an agent intended to reduce the processes of epidermal differentiation and/or to promote the regeneration and/or renewal of the skin.

The mosaic-effect tensing agent may be intended, in accordance with the invention, for one or more conditions selected from those mentioned above.

The effective amount of at least one mosaic-effect tensing agent that is present in the composition is intended to reduce the processes of epidermal differentiation and/or to promote the regeneration and/or renewal of the skin.

The biological effect of the tensors demonstrated by the Applicant is a durable biological effect, in contrast to the immediate visible effect which has been known to date for tensing agents.

The durable biological effect is explained in particular by the fact that the mechanical stresses induced by the tensors within the stratum corneum affect the whole of the cutaneous tissue: within the epidermis, the keratinocytes, step by step, transmit the mechanical stresses to their neighbouring cells; within the dermis, the mechanical stress is transmitted to the cells, in particular the fibroblasts, via the extracellular matrix. The mechanical stresses are thus transmitted within the cells via mechanoreceptors which convert this mechanical signal into biochemical signals, which in turn are responsible for gene-expression modulations that are involved in the overall homeostasis of the tissue.

The durable or remanent effect obtained on the homeostasis of the skin and in particular the thickness of the skin may be optimized by twice-weekly or, better still, daily application of a composition according to the invention.

This durable or remanent effect via repeated application of the mosaic-effect tensing agents may be explained as follows: the modulation of the physiology of a tissue by the tensors is the consequence, at the molecular level, of the modulation in the synthesis of proteins involved, on the one hand, in the differentiation and the proliferation of keratinocytes and fibroblasts and, on the other hand, in the organization of the matrix. For the molecular modifications to have a consequence at the macroscopic level it is necessary for them to accumulate. Consequently, as for all biological stimulations, it is necessary to repeat the stimulation by the tensors in order for them to lead to a restructuring of the cutaneous tissue.

The effective amount of at least one mosaic-effect tensing agent that is applied to the surface layers of the skin is intended in particular to regulate the expression of genes involved in the homeostasis of the skin via the transmission of mechanical tensions involving the activation of mechanoreceptors.

By “mosaic-effect tensing agent” that can be used in accordance with the invention is meant a compound liable to have a tensing effect, in other words able to stretch the skin, and a mosaic effect, as will be described hereinafter.

Generally speaking, a tensing agent, according to the invention, refers to any compounds which are soluble or dispersible in water at a temperature ranging from 25° C. to 50° C. at a concentration of 7% by weight in water or at the maximum concentration at which they form a medium of homogeneous appearance, and which at this concentration of 7% or at this maximum concentration in water produce a retraction of more than 15% in the test described below.

The maximum concentration at which they form a medium having a homogeneous appearance is determined to +10% and preferably to +5%.

By ‘medium having a homogeneous appearance’ is meant a medium which does not exhibit aggregates visible to the naked eye.

To determine said maximum concentration, the tensing agent is added gradually to water, with stirring using a deflocculator device, at a temperature ranging from 25° C. to 50° C., and then the mixture is kept with stirring for an hour. Inspection then takes place after 24 hours to determine whether the mixture thus prepared has a homogeneous appearance (absence of aggregates visible to the naked eye).

The tensing effect may be characterized by an in vitro retraction test.

Beforehand, and as described above, a homogeneous mixture of the tensing agent is prepared in water, at the concentration of 7% by weight or at the maximum concentration defined above.

30 μl of the homogeneous mixture are placed on a rectangular test specimen (10×40 mm, hence having an initial width W₀ of 10 mm) of elastomer having a modulus of elasticity of 20 MPa and a thickness of 100 μm.

After 3 h of drying at 22±3° C. and 40±10% relative humidity (RH), the elastomer test specimen exhibits a retracted width, recorded as W₃ₕ, owing to the tension exerted by the applied tensing agent.

The tensing effect (TE) of said agent is then quantified as follows:

‘TE’=(W₀−W₃ₕ/W₀)×100 in %

where W₀=initial width, 10 mm

and W₃ₕ=width after 3 h of drying

Furthermore, the tensing agent according to the invention must be a mosaic-effect tensing agent.

By “mosaic-effect tensing agent” is meant, in accordance with the invention, an agent which, when applied to a glass plate, dries to form a tessellated deposit, it being possible for the size and shape of its constituent tessellae to depend on their location relative to the edges of the deposit.

By “tessellated deposit” is meant more precisely a discontinuous deposit made up of a multiplicity of small individualized domains or microdomains.

The tessellae or microdomains are generally small in size. This size may range from 0.1 mm² to several mm².

In particular a mosaic deposit of this kind cannot be peeled or detached from the substrate, in contrast to continuous or semi-continuous deposits which adhere to the substrate and which can be detached or peeled either in a single piece or in two or more relatively large-sized strips.

Moreover, a mosaic deposit of this kind generally exhibits low resistance to water; in other words, on contact with water, the deposit breaks up.

A mosaic deposit of this kind according to the invention is shown in FIG. 1.

The tessellated or mosaic appearance of the deposit results in particular from the fact that the stresses developed by these tensing agents in the course of drying are greater than the forces of cohesion (rigidity) of the deposit.

From a mechanical standpoint, deposits of this kind formed by these mosaic-effect tensing agents may be characterized by their property of fracturing under stresses, which is evaluated for example in the mechanical strength test described below.

In particular, the mosaic-effect tensing agents according to the invention form deposits characterized advantageously by a breaking energy of between 0 and 20 J/m² (preferably equal to 0) and a breaking deformation of between 0 and 0.2 mm in the mechanical strength test described below.

Said mechanical strength test consists for example in applying compressive stress to breaking point to the tensing agent at the surface of a flexible and deformable foam.

The tensing agent is deposited at a concentration of 7% by weight in water or at the maximum concentration by weight at which it forms, in water at a temperature ranging from 25° C. to 50° C., a medium having a homogeneous appearance.

The use of this foam substrate makes it possible to apply a substantial deformation to the surface-deposited tensing agent, and hence makes it possible to quantify its breaking strength. The mechanical compressive stress is exerted by means of a cylindrical punch 1 mm in diameter, the rate of displacement of the punch being 0.1 mm/s.

The test is carried out using a TA-XT2i texture analyser sold by Stable Micro System. In this way a curve of force F (in N) as a function of displacement d (in mm) is obtained, from which it is possible to determine the breaking point of the material (tensing agent) or breaking energy W_{break}, expressed in J/m², as shown in FIG. 2.

The breaking energy W_{break} expressed in J/m² corresponds to the area under the curve F=f(d) obtained at the value of the displacement for which a discontinuation F_{break} (N) is observed.

The mosaic-effect tensing agents used in accordance with the invention are advantageous relative to other tensing agents, which form a continuous or semicontinuous deposit which adheres to a flexible substrate such as the skin, in that they allow better distribution, over the whole surface area of the skin, of the tensions exerted, relative to a continuous or semicontinuous deposit.

This is because, in the case of a continuous or semicontinuous deposit which adheres to a flexible substrate, the tensions develop in the substrate solely at the periphery of the solid deposit.

In contrast, in the case of a mosaic deposit according to the invention, with the same surface area as the above deposit, the tensions develop at the periphery of each independent tessella of the deposit, thereby considerably increasing the surface area subject to the tensions.

By virtue of these tensions being more effectively distributed over the entire surface area of the skin on which the deposit is formed, the Applicant has been able to show that the biological effects are obtained much more rapidly: starting from the first few hours after application of said mosaic-effect tensing agent to the skin, and in particular after 48 hours.

A mosaic-effect tensing agent of this kind in accordance with the invention may be selected from (i) mineral tensing agents, (ii) tensing polymers, and mixtures thereof.

The mineral tensing agents may be selected from:

a) mixed silicates;
b) colloidal particles of inorganic fillers;
and mixtures thereof.

The mosaic-effect tensing polymers may be selected from synthetic polymers and mixtures thereof.

A person skilled in the art will know how to select, from the chemical categories listed above, the materials conforming to the tensing test as described above.

These various categories of tensing agents will now be described.

### (i) Mineral Tensing Agents

Mixed Silicates

By this expression are meant all of the silicates of natural or synthetic origin which contain at least two different cations selected from alkali metals (for example Na, Li, K) or alkaline-earth metals (for example Be, Mg, Ca) and transition metals.

Preference is given to using phyllosilicates, namely silicates having a structure in which the SiO₄ tetrahedra are organized in sheets between which the metal cations are enclosed.

One class of silicates that is particularly preferred as tensing agents is that of the laponites. Laponites are magnesium lithium sodium silicates which have a layer structure similar to that of montmorillonites. Laponite is the synthetic form of the natural mineral known as hectorite. Use may be made, for example, of the laponite sold under the name Laponite XLS or Laponite XLG by Rockwood.

Colloidal Particles of Mineral Filler

By “colloidal particles” are meant particles in dispersion in an aqueous, aqueous-alcoholic or alcoholic medium, preferably an aqueous medium, which have a number-average diameter of between 0.1 and 100 nm, preferably between 3 and 30 nm.

The colloidal particles according to the invention have no thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2θ angle.

These particles are generally prepared by a sol-gel process and are therefore different in particular from particles of fumed silica, which undergo agglomeration in water to form larger aggregates.

The colloidal particles of mineral filler which can be used in accordance with the invention are generally selected from colloidal particles of silica, cerium oxide, zirconium oxide, alumina, calcium carbonate, barium sulphate, calcium sulphate, zinc oxide and titanium dioxide, colloidal particles of platinum, mixed colloidal particles such as, for example, titanium dioxides with one or more coatings, such as titanium dioxide with a silica coating. In the composition according to the invention preference will be given to using colloidal silicas or colloidal silica-alumina composite particles.

### Colloidal Particles of Silica

By colloidal silicas are meant, for the purposes of this application, colloidal particles of silica in dispersion in an aqueous, aqueous-alcoholic or alcoholic medium. The colloidal particles of silica have a diameter ranging from 0.1 to 100 nm and preferably from 3 to 30 nm. These particles are present in the form of aqueous dispersions and do not have any thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2θ angle.

Colloidal silicas which can be used in the composition according to the invention include, for example, those sold by Catalysts & Chemicals under the names Cosmo S-40 and Cosmo S-50.

### Colloidal Silica-Alumina Composite Particles

The colloidal particles of mineral fillers which can be used according to the invention may also be selected from colloidal silica-alumina composite particles. By silica-alumina composite is meant particles of silica in which atoms of aluminium have been partly substituted for atoms of silica. By colloidal particles are meant, for the purposes of this application, colloidal particles in dispersion in an aqueous, aqueous-alcoholic or alcoholic medium. The colloidal silica-alumina composite particles have a diameter ranging from 0.1 to 100 nm and preferably from 3 to 30 nm. These particles are present in the form of aqueous dispersions and do not have any thickening property in water, alcohol, oil and all other solvents. At a concentration greater than or equal to 15% by weight in water, the viscosity of the solutions thus obtained is less than 0.05 Pa·s for a shear rate of 10 s⁻¹. The measurements are made at 25° C. using a Haake RheoStress RS150 rheometer in cone/plate configuration, the measurements of the measuring cone being as follows: 60 mm diameter and 2θ angle.

At a pH of 7, the colloidal silica-alumina composite particles according to the invention have a zeta potential of less than −20 mV and preferably less than −25 mV. The measurements are made at 25° C. using a Coulter Scientific Instrument DELSA 440SX apparatus.

Colloidal silica-alumina composite particles which can be used in the compositions according to the invention include, for example, those sold by Grace under the names Ludox AM, Ludox AM-X 6021, Ludox HSA and Ludox TMA.

The preferred mineral tensing agents in accordance with the invention are colloidal particles selected from colloidal particles of silica and colloidal silica-alumina composite particles. Mention will be made in particular of those sold under the names Cosmo S-40 and Cosmo S-50 and those sold under the names Ludox AM, Ludox HSA and Ludox TMA.

The preferred colloidal particles of inorganic fillers are selected from colloidal particles of silica.

### (ii) Tensing Polymers

The tensing polymers used in accordance with the invention are generally synthetic polymers.

The person skilled in the art will know to select, from the chemical categories of polymers listed below, the polymers which produce the above-described mosaic effect and comply with the mechanical strength test as described above.

In particular the polymers according to the invention form deposits characterized by a breaking energy of between 0 and 20 J/m², and a deformation at break of between 0 and 0.2 mm, in the mechanical strength test described above.

The synthetic polymers used according to the invention may be in solution or in suspension in a polar or apolar (latex) liquid, particularly in aqueous solution or aqueous dispersion, or in a dry form which is redispersible in a cosmetic solvent.

The synthetic polymers which can be used as a tensing agent may be selected from acrylic polymers. The synthetic polymers according to the invention may in particular be selected from interpenetrating polymer networks (IPNs).

These polymers may take the form, in particular, of random linear copolymers, of interpenetrating polymer networks (IPNs), and of block polymers. Irrespective of its nature, the synthetic polymeric tensing agent may have a weight-average mass Mw ranging from 3000 to 1 000 000 Da.

### Random Linear Copolymers

The random linear copolymers which are tensors in the sense of the present invention are selected from random copolymers having a linear ethylenic main chain with a molecular weight of less than 600 000 Da (g/mol), preferably a weight-average molecular weight of between 15 000 and 600 000 g/mol, and contain at least 70% of a monomer with a glass transition temperature, Tg, greater than 40° C. (preferably >60° C.) whose corresponding homopolymer is insoluble in water at 25° C., and at least one ionic hydrophilic monomer. This copolymer may also contain a non-majority monomer with a Tg less than 40° C.

These copolymers generally exhibit an overall glass transition temperature greater than or equal to 45° C.

Preference is given to all copolymers composed of:
70% to 90% by weight of at least one aryl or alkyl acrylate and (or) at least one aryl or alkyl methacrylate cited in the list below and (or) styrene
10% to 30% by weight of (meth)acrylic acid.

List of preferred alkyl (meth)acrylates: benzyl acrylate, cyclohexyl acrylate, tert-butyl acrylate, isobornyl acrylate and norbornyl acrylate, methyl, ethyl, isobutyl, cyclohexyl, benzyl, tert-butyl, isobornyl and norbornyl methacrylate, preferably methyl methacrylate and cyclohexyl methacrylate.

Among the abovementioned polymers preference will be given particularly to:
copolymers of methyl methacrylate/methacrylic acid; copolymers of methyl methacrylate/acrylic acid, said copolymers containing between 70% and 90% by weight of methyl methacrylate;
copolymers of ethyl methacrylate/methacrylic acid; copolymers of ethyl methacrylate/acrylic acid, said copolymers containing between 70% and 90% by weight of ethyl methacrylate;
copolymers of isobutyl methacrylate/methacrylic acid; copolymers of isobutyl methacrylate/acrylic acid, said copolymers containing between 70% and 90% by weight of isobutyl methacrylate;
copolymers of benzyl methacrylate/methacrylic acid; copolymers of benzyl methacrylate/acrylic acid, said copolymers containing between 70% and 90% by weight of benzyl methacrylate;
copolymers of benzyl acrylate/methacrylic acid; copolymers of benzyl acrylate/acrylic acid, said copolymers containing between 70% and 90% by weight of benzyl acrylate;
copolymers of cyclohexyl methacrylate/methacrylic acid; copolymers of cyclohexyl methacrylate/acrylic acid, said copolymers containing between 70% and 90% by weight of cyclohexyl methacrylate;
copolymers of cyclohexyl acrylate/methacrylic acid; copolymers of cyclohexyl acrylate/acrylic acid, said copolymers containing between 70% and 90% by weight of cyclohexyl acrylate;
copolymers of tert-butyl methacrylate/methacrylic acid; copolymers of tert-butyl methacrylate/acrylic acid, said copolymers containing between 70% and 90% by weight of tert-butyl methacrylate;
copolymers of tert-butyl acrylate/methacrylic acid; copolymers of tert-butyl acrylate/acrylic acid, said copolymers containing between 70% and 90% by weight of tert-butyl acrylate;
copolymers of isobornyl methacrylate/methacrylic acid; copolymers of isobornyl methacrylate/acrylic acid, said copolymers containing between 70% and 90% by weight of isobornyl methacrylate;
copolymers of isobornyl acrylate/methacrylic acid; copolymers of isobornyl acrylate/acrylic acid, said copolymers containing between 70% and 90% by weight of isobornyl acrylate;
copolymers of norbornyl methacrylate/methacrylic acid; copolymers of norbornyl methacrylate/acrylic acid, said copolymers containing between 70% and 90% by weight of norbornyl methacrylate;
copolymers of norbornyl acrylate/methacrylic acid; copolymers of norbornyl acrylate/acrylic acid, said copolymers containing between 70% and 90% by weight of norbornyl acrylate; and
copolymers of styrene/methacrylic acid; copolymers of styrene/acrylic acid, said copolymers containing between 70% and 90% by weight of styrene.

The copolymers in accordance with the present invention are in the form of a dispersion in a polar liquid. These copolymers are dispersed in water after neutralization with a base.

A preferred copolymer according to the invention is selected from copolymers of methyl methacrylate/methacrylic acid, said copolymers containing between 70% and 90% by weight of methyl methacrylate.

### Interpenetrating Polymers or IPNs

An “interpenetrating polymer network” for the purposes of the present invention means a mixture of two inter-meshed polymers obtained by simultaneous polymerization and/or crosslinking of two types of monomers, the resulting mixture having a single glass transition temperature. Examples of IPNs that are suitable for employment in the present invention, and the process for preparing them, are described in patents U.S. Pat. No. 6,139,322 and U.S. Pat. No. 6,465,001, for example. Preferably the IPN according to the invention comprises at least one acrylic polymer and, more preferably, it further comprises at least one polyurethane or a copolymer of vinylidene fluoride and hexafluoropropylene. According to one preferred embodiment the IPN according to the invention comprises a polyurethane and an acrylic polymer. IPNs of this kind are in particular those of the Hybridur series which are available commercially from Air Products. One particularly preferred IPN is in the form of an aqueous dispersion of particles having a weight-average size of between 90 and 110 nm and a number-average size of approximately 80 nm. This IPN preferably has a glass transition temperature, Tg, which ranges from approximately −60° C. to +100° C. An IPN of this type is sold in particular by Air Products under the trade name Hybridur 875. Other IPNs suitable for use in the present invention are referenced Hybridur X01602 and Hybridur 580.

Other IPNs suitable for employment in the present invention include IPNs composed of a mixture of a polyurethane with a copolymer of vinylidene fluoride and hexafluoropropylene. These IPNs can be prepared in particular as described in patent U.S. Pat. No. 5,349,003. As a variant, they are available commercially in the form of a colloidal dispersion in water, in a ratio of the fluoro copolymer to the acrylic polymer of between 70:30 and 75:25, under the trade names KYNAR RC-10, 147 and KYNAR RC-10, 151 from ATOFINA.

### Block Polymer

As a variant, the synthetic polymeric tensing agents which can be used in the composition according to the invention may be block polystyrene (PS)-polyethyl acrylate (PEA) polymers.

Very generally, a block copolymer is a polymer composed of at least two distinct homopolymers composed solely of monomers A and B respectively. Thus the blocks according to the invention are, respectively, polystyrene (PS) and polyethyl acrylate (PEA) blocks.

In the context of this variant, the polymer may be a triblock polymer of type PS-PEA-PS or else a multiblock polymer of type PS-[PEA-PS]n, or PEA-[PS-PEA]n, where n is a positive integer and is preferably 1. Advantageously these block polymers are linear copolymers. The molecular weight of this polymer is preferably greater than 10 000 daltons, and more preferably greater than 50 000 daltons. The ratio by weight of the PS and PEA monomers may be defined such that PS/PEA is greater than 1 and preferably such that PS/PEA is greater than 5.

Mention may be made of the triblock polymer PS(30 000)-PEA(10 000)-PS(30 000), which is most particularly suitable for the implementation of the invention. This particularly advantageous block copolymer is a triblock copolymer comprising:
a first block containing units deriving from styrene, having a number-average molecular mass of 30 000 g/mol;
a second block composed of units deriving from ethyl acrylate, having a number-average molecular mass of 10 000 g/mol;
a third block containing units deriving from styrene, having a number-average molecular mass of 30 000 g/mol.

A copolymer conforming to the definition given above may be a copolymer for which the first block and/or the third block and, preferably, the first block and the third block comprise, in addition to the units deriving from styrene, units deriving from methacrylic acid, for example, in a mass ratio (styrene/methacrylic acid) of 98/2.

The synthetic copolymers used according to the invention may also, as a variant, be composed of a random polystyrene polyethyl acrylate copolymer. The ratio by weight of the PS and PEA monomers is defined such that PS/PEA>1 and preferably such that PS/PEA>5.

The synthetic polymer used in accordance with the invention is preferably selected from acrylic copolymers composed (a) of 70% to 90% by weight of a copolymer of at least one alkyl acrylate and/or at least one alkyl methacrylate and/or of styrene and (b) from 10% to 30% by weight of a copolymer of at least one ionic hydrophilic monomer.

In particular the synthetic polymer is an ethylenic copolymer.

Another preferred synthetic polymer is an interpenetrating polymer network-type polymer, in particular an interpenetrating polymer network comprising a polyurethane polymer and an acrylic polymer.

More preferably still, use will be made of an interpenetrating polymer comprising a polyurethane and an acrylic polymer.

The invention also relates to the cosmetic use of a composition comprising, in a physiologically acceptable medium, at least one interpenetrating polymer network for promoting and/or improving the elasticity and/or firmness and/or tonicity of the skin.

It also relates to the cosmetic use of a composition comprising, in a physiologically acceptable medium, at least one interpenetrating polymer network for promoting the regeneration and/or reorganization of the extracellular matrix.

The invention also relates to the cosmetic use of a composition comprising, in a physiologically acceptable medium, at least one interpenetrating polymer network for promoting the regeneration and/or renewal of the skin.

In particular, the interpenetrating polymer network comprises a polyurethane polymer and an acrylic polymer.

The mosaic-effect tensing agent will be present in the composition in an amount effective for obtaining the required biological effect according to the invention.

By way of example the mosaic-effect tensing agent may be included in the composition according to the invention in an amount ranging from 0.01% to 30% by weight of active ingredient, preferably from 1% to 30% by weight of active ingredient, relative to the total weight of the composition.

In particular it will be possible to use an effective amount of tensing agent ranging from 2% to 30% by weight, in particular from 3% to 20%, preferably from 4% to 20% by weight of active ingredient relative to the total weight of the composition, in particular from 3% to 7%, or for example an amount between 6% and 10% by weight of active ingredient relative to the total weight of the composition.

According to one particular embodiment an effective amount of tensing agent will be used of 3% to 20% by weight of active ingredient relative to the total weight of the composition.

By “active ingredient” the intention is to exclude the medium in which the tensing agent is optionally solubilized or in dispersion in its commercial form, as for example in the case of dispersions of colloidal particles.

The composition according to the invention comprises a physiologically acceptable medium, in other words a medium which is compatible with the skin of the face and/or body. It is preferably a cosmetically acceptable medium, in other words a medium which has a colour, odour and feel that are pleasant and which does not give rise to any unacceptable discomfort (stinging, tautness, redness) that might dissuade the consumer from using this composition.

The composition according to the invention may be a bodycare or facial-care composition or a makeup composition.

The composition according to the invention may be in any of the formulated forms conventionally used for topical application, and particularly in the form of dispersions of the aqueous gel or lotion type, emulsions with a liquid or semi-liquid consistency of the milk type, obtained by dispersing a fatty phase in an aqueous phase (O/W) or conversely (W/O), or of suspensions or emulsions with a soft, semi-solid or solid consistency, of cream or gel type, or in the form of a serum or stick, or else of multiple emulsions (W/O/W or O/W/O). These compositions are prepared in accordance with the customary methods.

Oils which can be used in the composition according to the invention include the following:
hydrocarbon oils of animal origin, such as perhydrosqualene;
hydrocarbon oils of vegetable origin, such as liquid triglycerides of fatty acids containing 4 to 10 carbon atoms, or else, for example, vegetable oils such as apricot kernel oil and shea butter oil;
synthetic esters and ethers, especially those of fatty acids, such as the oils of formulae R¹COOR² and R¹OR² in which R¹ represents the residue of a fatty acid containing 8 to 29 carbon atoms and R² represents a branched or unbranched hydrocarbon chain containing 3 to 30 carbon atoms;
linear or branched hydrocarbons of mineral or synthetic origin, such as volatile or non-volatile liquid paraffins and derivatives thereof, isohexadecane, isododecane, Vaseline, polydecenes, hydrogenated polyisobutene such as Parléam® oil;
natural or synthetic essential oils;
branched fatty alcohols having 8 to 26 carbon atoms, such as octyldodecanol;
partially hydrocarbon-modified and/or silicone-modified fluoro oils like those described in document JP-A-2-295912;
silicone oils such as volatile or non-volatile polydimethylsiloxanes (PDMSs) having a linear or cyclic silicone chain which are liquid or pasty at ambient temperature, especially cyclopolydimethylsiloxanes (cyclomethicones) such as cyclohexasiloxane and cyclopentasiloxane; polydimethylsiloxanes containing alkyl, alkoxy or phenyl groups pendantly or at the end of a silicone chain, these groups having 2 to 24 carbon atoms; phenyl silicones such as phenyltrimethicones, phenyldimethicones, phenyltrimethylsiloxydiphenylsiloxanes, diphenyldimethicones, diphenylmethyldiphenyltrisiloxanes, 2-phenylethyl trimethylsiloxysilicates, and polymethylphenylsiloxanes; and
mixtures thereof.

The other fatty substances which may be present in the oily phase are, for example, fatty acids containing 8 to 30 carbon atoms, such as stearic acid, lauric acid, palmitic acid and oleic acid; linear fatty alcohols such as cetyl alcohol and/or stearyl alcohol; pasty fatty substances such as lanolin; waxes; and gums such as silicone gums (dimethiconol).

These fatty substances may be selected variously by the skilled worker in order to prepare a composition having the desired properties of, for example, consistency or texture.

This composition may further comprise various adjuvants commonly used in the field of cosmetology, such as emulsifiers, including esters of fatty acids and of polyethylene glycol, esters of fatty acid and of sorbitan which are optionally polyoxyethylenated, polyoxyethylenated fatty alcohols and the esters or ethers of fatty acid and of sugars such as sucrose or glucose; fillers; preservatives; sequestrants; fragrances; and thickeners and/or gelling agents, such as homopolymers and copolymers of acrylic acid, homopolymers and copolymers of acrylamide and/or of 2-acrylamido-2-methylpropanesulphonic acid (AMPS), modified AMPS (Aristoflex LNC and SNC) products, and xanthan gum.

Fillers include, for example, particles of polyamide (Nylon) in spherical or microfibre form; microspheres of polymethyl methacrylate; ethylene-acrylate copolymer powders; expanded powders such as hollow microspheres and, in particular, the microspheres formed from a terpolymer of vinylidene chloride, acrylonitrile and methacrylate which are sold under the name Expancel; powders of natural organic materials such as powders of starch, particularly of maize starch, wheat starch or rice starch, crosslinked or non-crosslinked, such as powders of starch crosslinked with octenylsuccinic anhydride; silicone resin microbeads such as those sold under the name Tospearl by Toshiba Silicone; silica; metal oxides such as titanium dioxide or zinc oxide; mica; hollow hemispherical particles of silicone such as NLK506 sold by Takemoto Oil and Fat; and mixtures thereof.

The skilled worker will of course take care to select this or these optional additional compounds and/or their amount in such a way that the advantageous properties of the composition according to the invention are not, or not substantially, adversely affected by the intended addition.

The composition according to the invention is applied in accordance with the typical techniques, for example by application of creams, gels, serums or lotions to the skin it is intended to treat, in particular the skin of the body, face and/or neck.

The invention also provides a cosmetic method of treatment of dull and/or poorly defined complexion, characterized in that a composition comprising, in a physiologically acceptable medium, an effective amount of at least one mosaic-effect tensing agent is applied to people who have a dull and/or poorly defined complexion.

Likewise part of the context of the invention is a cosmetic method of treatment of soft and/or flaccid skin, characterized in that a composition comprising, in a physiologically acceptable medium, an effective amount of at least one mosaic-effect tensing agent is applied to people who have soft and/or flaccid skin or to areas of the body exhibiting a loss of elasticity and/or firmness.

In particular it will be possible to apply the composition to the face and/or neck, stomach and thighs.

Advantageously, and in order to obtain a remanent effect of the tensing agents on the homeostasis of the skin, it will be possible to apply the composition according to the invention twice-weekly and, more preferably, daily, in the morning and/or the evening.

The invention also provides a cosmetic method of treatment of the skin that comprises the application to the skin, in accordance with a frequency of at least twice per week, of a composition comprising at least 3% by weight, relative to the total weight of the composition, of at least one mosaic-effect tensing agent.

In particular the composition will comprise from 3% to 30% by weight, and preferably from 3% to 7% by weight, relative to the total weight of the composition, of at least one mosaic-effect tensing agent.

More preferably still the composition is applied to the skin at a frequency of at least once per day.

The effect of repeated application to the skin of tensing agents according to the invention on the mechanical properties of the skin, and in particular on the firmness, elasticity and extensibility of the skin, were also able to be confirmed and/or evaluated in vivo by instrumental devices such as those described below.

### The Torquemeter:

This apparatus aims to measure the variations in extensibility, in firmness/elasticity and in tonicity of the skin. The apparatus imposes a torsion in the plane of the skin for a given time; the skin is then subjected to a stretching which corresponds to its extensibility (Ue); after the arrest of the torsion, the skin regains its initial “shape”, and thus its tonicity (Ur) is evaluated. The firmness/elasticity of the skin corresponds to the ratio Ur/Ue (tonicity over extensibility).

### The Cutometer:

The cutometer is a suction apparatus composed of a cylinder from 1 to 3 mm in diameter which is applied to the skin. Drawing up the skin by means of a pump connected to the cylinder induces a vertical displacement of the skin, which makes it possible to evaluate the mechanical properties of the skin.

### The Densiscore:

The densiscore is an apparatus allowing the density of the skin to be measured. It subjects the skin locally to a mechanical stress which gives rise to folds, the number and amplitude of which are directly associated with the cutaneous density. Evaluation by trained experts of the profile of the skin subjected to the densiscore allows an evaluation of the density of the skin.

Moreover, the effect of repeated application to the skin of tensing agents according to the invention on the reorganization of the extracellular matrix may also be confirmed and/or evaluated in vivo by a technique of ultrasound echography.

The degradation and/or disorganization of the extracellular matrix at the level of the papillary dermis is a major cause of aging of the skin. It is partly responsible for the appearance of wrinkles and for the loss of density, firmness and extensibility of the skin. This degradation and/or disorganization of the extracellular matrix is even more visible in people who have mature (>40 years) or even very mature (>60-65 years) skin.

Ultrasound echography makes it possible to obtain 2D or 3D images of cutaneous tissues. The intensity of the echoes reflected provides information on the nature, density and organization of the constituents of the dermis. In particular it evidences differences between the superficial dermis or papillary dermis and the deep dermis or reticular dermis. This technique therefore makes it possible to evaluate the effect of the repeated application of tensing agents according to the invention on the reorganization and restructuring of the papillary dermis.

The invention will now be described with reference to the following examples, which are given by way of illustration and not of limitation. In these examples, unless indicated to the contrary, the amounts are expressed in weight percentages.

### FIGURES

FIG. 1: Example of mosaic deposition of Hybridur 875 (magnification ×30).

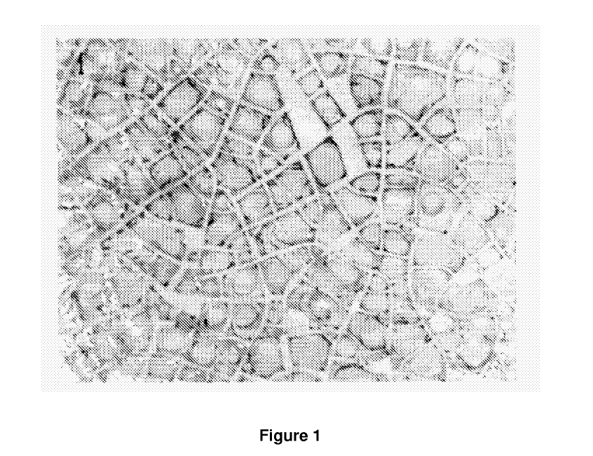

FIG. 2: Example of a curve of force as a function of displacement.

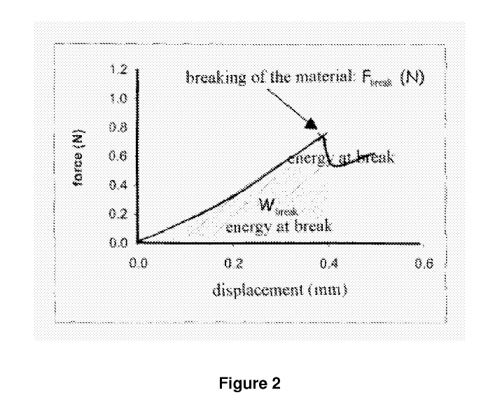

FIG. 3: Schematic representation of an electron micrograph showing the effect of Hybridur 875 on the reorganization of collagen fibrils in the extracellular matrix.

### EXAMPLES

### Example 1

### Demonstration of the Biological Effects of the Tensors

a) Effect on the Differential Expression of Genes

The biological effects of the tensing agents were demonstrated after application to Episkin® reconstructed epidermides.

### Culture Conditions of Reconstructed Epidermides

The Episkin® reconstructed epidermides used were obtained at d 15. They were placed in a maintenance medium for 8 hours. They were then transferred to a DMEM/Ham F12 medium devoid of EGF, of pituitary extract and of foetal calf serum. The epidermides were placed in this medium for 24 hours in order to equilibrate.

### Preparation of the Tensor: Ethylenic Copolymer of Methyl Methacrylate/Methacrylic Acid Copolymer Type

### Step 1: Synthesis of the Polymer

A jacketed 2 l reactor was charged with 1 g of Trigonox 21S (tert-butyl peroxy-2-ethylhexanoate) and 200 g of methyl ethyl ketone. The mixture was heated at reflux for 1 h. After 1 h a mixture of 170 g of methyl methacrylate and 30 g of methacrylic acid was added dropwise over a time of 1 h. The colourless mixture became viscous. Heating was interrupted 6 h after the addition of the monomers.

Composition by NMR: methyl methacrylate 85.1%, methacrylic acid 14.9%

Mass by GPC in THF (polystyrene standards): Mw=98772; Mn=61261; Mw=105698 lp=1.7

### Step 2: Dispersion of the Polymer in Water

The above reaction mixture was admixed with 200 g of methyl ethyl ketone and heated to 60° C. 30.86 g of 2-amino-2-methylpropanol and 1200 g of water were added dropwise. The volatile solvents were evaporated by heating to 100° C. This gave a transparent yellow aqueous dispersion.

One hundred microlitres of an aqueous dispersion of this ethylenic copolymer were then applied to the Episkins in this culture medium and left in contact with the epidermides for 24 hours in a chamber thermostated at 37° C. and 40% relative humidity. At the end of this period, the epidermides were withdrawn and extracted for the cDNA array studies.

### Dedicated Analysis by Minichips

Gene expression analysis was performed by using standard DNA arrays containing 159 genes that were dedicated to the search and adapted for screening. These minichips were produced on a nylon support by fixing marker-specific cDNAs involved in the regulation of the physiology of keratinocytes and of the skin in general. The analysis is performed by an in-house optimized and miniaturized technique based on the use of mRNA and of labelling with phosphorus 33 (P33).

Schematically, the mRNAs of the cells were extracted and purified with the aid of trireagent, the mRNA of each culture is “reverse” transcribed using oligo dT and a P33-labelled deoxynucleotide triphosphate. Multiple cDNA “target” labelled sequences were therefore produced for each Episkin® reconstructed epidermis. These targets were then hybridized, under optimized conditions, with cDNA “probes” in excess, fixed on the membranes. After washing, the quantity of labelled target is visualized by autoradiography and by direct counting on a PhosphorImager. The membranes are analysed by the Imagequant software.

The results are expressed in relative expression units. The levels of expression were corrected 1) for the average background noise present on each membrane and 2) for the differences in labelling intensity of the different probes used. This correction is carried out on the basis of differences in labelling intensity of the reference genes. The average of the counting results of “housekeeping gene” markers, whose expression is generally considered to be stable, was taken as a reference for quantifying, relatively, the expression of the other markers.

The significance limit was set at 180% of the untreated control for a stimulating effect and at 50% of the control for a repressive effect.

### Results

### Modulation of the Expression of Genes Involved in the Differentiation of Keratinocytes:

25 genes out of the 159 present on the dedicated mini-chips were modulated by the tensing agent. These genes intervene in the regulation of the physiology of keratinocytes and/or of fibroblasts.

The table below presents all of the results obtained on the effect of the tensors on the expression of these genes.

| | | | | | % variation |
|---|---|---|---|---|---|
| | | | | | of the |
| | | | | | expression |
| | | | | Growth | of the genes |
| | | Differen- | Metallo- | factors, | relative to the |
| Abbrevi- | | tiation | protein- | cytokines, | untreated |
| ation | Name of gene | markers | ases | receptors | control |
| AZGP1 | alpha-2- | | | | 21 |
| | glycoprotein 1, | | | | |
| | zinc | | | | |
| B2M | beta-2-micro- | | | | 486 |
| | globulin | | | | |
| CDSN | corneodesmosin | x | | | 51 |
| CST6 | cystatin 6 | x | | | 35 |
| CK1 | cytokeratin 1 | x | | | 980 |
| KRT19 | type I | x | | | 293 |
| | cytoskeletal 19 | | | | |
| | keratin | | | | |
| KRT2E | type II | x | | | 1163 |
| | cytoskeletal 2 | | | | |
| | epidermal keratin | | | | |
| | (KRT2E; KRT2A) | | | | |
| KRT6A | type II | x | | | 386 |
| | cytoskeletal 6 | | | | |
| | keratin: K6A | | | | |
| | keratin (KRT6A) | | | | |
| LOR | loricrin | x | | | 50 |
| NICE-1 | NICE-1 protein | x | | | 58 |
| CRBP1 | retinol-binding | x | | | 326 |
| | protein I | | | | |
| SPRL1B, | SPRL “small | x | | | 20 |
| XP5 | proline rich- | | | | |
| | like” protein | | | | |
| | (epidermal | | | | |
| | differentiation | | | | |
| | complex) 1B or | | | | |
| | skin-specific | | | | |
| | protein (XP5) | | | | |
| SPRL6A, | small proline- | x | | | 37 |
| LEP16 | rich-like | | | | |
| | (epidermal | | | | |
| | differentiation | | | | |
| | complex) 6A or | | | | |
| | SPRL6A; or late | | | | |
| | envelope protein | | | | |
| | 16 | | | | |
| KLK7 | kallikrein 7 | x | | | 56 |
| SBS | suprabasin (SBS) | x | | | 33 |
| ZYX | zyxin | x | | | 53 |
| MMP3 | matrix | | x | | 37 |
| | metalloproteinase | | | | |
| | 3 (MMP3) | | | | |
| IL6 | interleukin-6 | | | x | 340 |
| IL8 | interleukin-8 | | | x | 259 |
| TLR1 | oll-like receptor 1 | | | x | 434 |
| TGFB1 | transforming | | | x | 437 |
| | growth factor | | | | |
| | beta 1 | | | | |
| HMOX1 | heme oxygenase 1 | | | | 60 |
| HSPCA | heat shock | | | | 1083 |
| | 90 kDa protein 1 | | | | |
| MT1H | metallothionein | | | | 52 |
| | IH | | | | |
| MIF | macrophage | | | | 54 |
| | migration | | | | |
| | inhibitory factor | | | | |

In particular, the ethylenic copolymer tested reduced the expression of a number of proteins which make up the stratum corneum, such as corneodesmosin and loricrin, by a factor of two, and suprabasin by a factor of 3, which suggests that the copolymer diminishes the process of terminal differentiation.

The acrylic copolymer increases, moreover, the expression of a number of proteins of the intermediate filaments of the cytoskeleton, the cytokeratins, which are found in particular in foetal epitheliums and regenerative epitheliums. After 24 h of treatment, the expression of cytokeratins 1 is increased by a factor of 10 and the expression of cytokeratin 19 is increased by a factor of 3. These two cytokeratins, although present in adult epidermides, have been described as being expressed in many types of epithelial tissues, in particular in non-stratified epitheliums and also foetal epitheliums (Haake et al., Exp Cell Res., 1997 Feb. 25; 231(1): 83-95). Also increased is the expression of cytokeratin 2E/A, by a factor of 10: this cytokeratin 2 has been described as being expressed both in an adult epidermis and in a foetal epidermis. Finally, the expression of cytokeratin 6 is increased by a factor of 4. This cytokeratin 6 has been described as being overexpressed in regenerative epidermides, particularly in the course of cicatrization (Mazzalupo et al., 2003 February; 226(2): 356-65), which suggests that, in the course of the tensions provided by the application of the acrylic copolymer, the epidermides adopt features of regenerative epidermides.

The ethylenic copolymer according to the invention reduces the expression of complexes necessary for the process of differentiation of keratinocytes, such as SPRL, also called LEP10, by a factor of 5, and in the case of SPRL6 by a factor of 2.

In parallel with this, these results show that the copolymer increases the expression of CRBP1, which is involved in the cellular response to retinol, by a factor of 3, which suggests that the tensions are able to sensitize the cells to retinol.

### Modulation of the Expression of TGFb.

The expression of TGFb is increased by a factor of 4. This cytokine increases the expression and the organization of all of the fibrillar collagens and also of the plasminogen activator of type I, PAIL, and reduces the expression of a number of enzymes involved in the degradation of the extracellular matrix, the metalloproteinases.

In the course of the tensions caused by the acrylic latex it will be possible for the induced TGFb to diffuse to the level of the dermis and thus to induce tissue repair. The increase in the expression of TGFb by the tensions may be considered as a testament to the sensitivity of the cells to the tensions brought about by the acrylic latex tensor.

### Reduction in the Expression of Metalloproteinases:

The ethylenic copolymer reduces the expression of metalloproteinase 3, which is involved on the one hand in the migration of cells and on the other hand in the degradation of the extracellular matrix. The ethylenic copolymer thus inhibits the degradation of the extracellular matrix and plays a part in cell migration.

### Reduction of Zyxin:

The ethylenic copolymer reduced the expression of zyxin, which is known to be located at the level of the adhesion complexes and to play a part in the cell morphology.

### Increase in the Response of Cells to Environmental Stresses

The ethylenic copolymer increased the expression of the chaperone protein HSP90A by a factor of 10. HSP90A proteins play a fundamental part during the process of maturing of the proteins. They regulate the conformation of kinase or of transcription factors and, consequently, control their activity and their degradation.

The totality of these data show that the mechanical tensions applied via an effective amount of the tensing agent according to the invention are sensed by the keratinocytes as a stimulus which leads to a slowdown in the process of differentiation of the epidermis; the modulation of the expression of the genes cited above appears to show, moreover, that the epidermis acquires a regenerative epidermal phenotype. These results indicate that topical application of an effective amount of at least one tensing agent makes it possible to promote the homeostasis of the skin and thus to increase the thickness of the skin and/or improve the mechanical properties of the skin and/or promote the radiance of the complexion.

Moreover, the increase of the expression of HSP90 suggests that the tensions will reinforce the capacity of the epidermis to combat the alteration of the homeostasis of the skin that is induced by environmental stresses.

### Example 2

### Demonstration of an Effect of the Tensors on the Radiance of the Complexion

A serum is prepared comprising the ethylenic copolymer described in Example 1:

| | | | |
|---|---|---|---|
| A- | Water | 50.45 | g |
| | Ammonium polyacryldimethyltauramide | 2.00 | g |
| | (Hostacerine AMPS) | | |
| | Preservatives | 0.85 | g |
| B- | Ethylenic copolymer of Example 1 | 46.70 | g |
| | (7% dispersion in water) | | |

The lightening and homogenizing effects and also the cosmetic aspects of this serum were evaluated on a panel of 6 subjects having normal/mixed skin. Following application of this serum in the evening for one month, a less poorly defined complexion was observed.

These results show that the biomechanical effect provided by the topical application of tensors to the skin, which is manifested in a reduction in epidermal differentiation and/or an increase in the regenerative power of the epidermis, makes it possible to enhance the appearance of the skin and in particular the radiance of the complexion.

### Example 3

### Mechanical Strength Testing of the Tensing Agents

The mechanical strength test consists in applying compressive stress to breaking point to the tensing agent under test at the surface of a flexible and deformable foam. The use of this foam support allows a substantial deformation to be imposed on the tensing agent under test, deposited at the surface, and therefore its breaking strength to be quantified.

The substrate is composed of a neoprene foam 13 mm in thickness.

The tensing agent, soluble or dispersible in water at a temperature ranging from 25° C. to 50° C. at a concentration of 7% by weight in water or at the maximum concentration by weight at which it forms in water at a temperature ranging from 25° C. to 50° C. a homogeneous medium visible with the naked eye, is deposited on this substrate to give, after drying for 24 h, a deposit with a thickness of 15 to 30 μm. The deposits were produced using a film-drawing device applying a wet film of 650 μm.

The mechanical compressive stress is exerted by means of a cylindrical punch with a diameter of 1 mm, the rate of displacement of the punch being 0.1 mm/s.

The test is carried out using a TA-XT2i texture analyser sold by Stable Micro System.

This gives a curve of force F (in N) as a function of displacement d (in mm), from which it is possible to determine the breaking point of the material (tensing agent) and the breaking energy W_{break} (J/m²) corresponding to the area under the curve F=f(d) at the breaking point F_{break} (N).

The totality of the results obtained are presented below:

| Compositions | d_{b} (mm) | W_{break} (J/m²) |
|---|---|---|
| Example A: Hybridur 875 (mosaic- | 0 | 0 |
| effect tensing agent of the | | |
| invention) | | |
| Example B: Eleseryl VGH8 | 0.4 | 40 |
| Example C: Flexan | 1.2 | 1600 |
| Example D: Avalure UR 405 | 1.7 | 1400 |
| Example E: Kytamer PCA | 1.5 | 3200 |

In particular, the mosaic-effect tensing agents according to the invention form deposits characterized advantageously by a breaking energy of between 0 and 20 J/m² (preferably equal to 0) and a breaking deformation of between 0 and 0.2 mm in this mechanical strength test.

### Example 4

### Demonstration of the Effect of the Tensors on the Reorganization of the Extracellular Matrix

### Principle of the Test

In order to define whether the application of tensing agents on the stratum corneum is able to induce modifications to the organization of the extracellular matrix, 100 μl of tensing agents, respectively Hybridur 875, sold by Air Products (at 15% by weight in water) and an acrylic tensor (ethylenic copolymer as prepared in Example 1, at 7% by weight in water), were applied to an Episkin® reconstructed skin model.

The reconstructed skin model, composed of human keratinocytes deposited on a support, often a dermis equivalent, and cultured under conditions such that they enter into a programme of differentiation leading ultimately to the formation of an epidermis equivalent, can be prepared according to the protocol described in Asselineau et al. (1987, Models in dermato., vol. III, Ed. Lowe & Maibach, 1-7).

Hybridur 875, sold by Air Products, is prepared in accordance with the description in patents U.S. Pat. No. 5,977,215 and U.S. Pat. No. 5,521,246.

The effect of these tensing agents on the dermis is observed after 2 h, 24 h and 48 h of application.

The analyses are carried out by two complementary imaging techniques: optical microscopy (multiphoton microscopy and optical microscopy in transmission of semi-fine sections) and electron microscopy (scanning electron microscopy).

Multiphoton microscopy allows rapid definition, without prior preparation of samples, of the actives which will exhibit an activity on the extracellular matrix, and on what timescale.

Its three-dimensional resolution makes it possible to determine the depth at which mechanical stimulation will cause dermal modifications.

When this first observation has been made, the samples are then analysed by scanning electron microscopy, which allows better resolution and hence allows the collagen fibrils to be individualized.

The modifications observed to the extracellular matrix are connected to modifications at the level of the fibroblasts by virtue of studies in optical microscopy in transmission of semi-fine sections.

### Results:

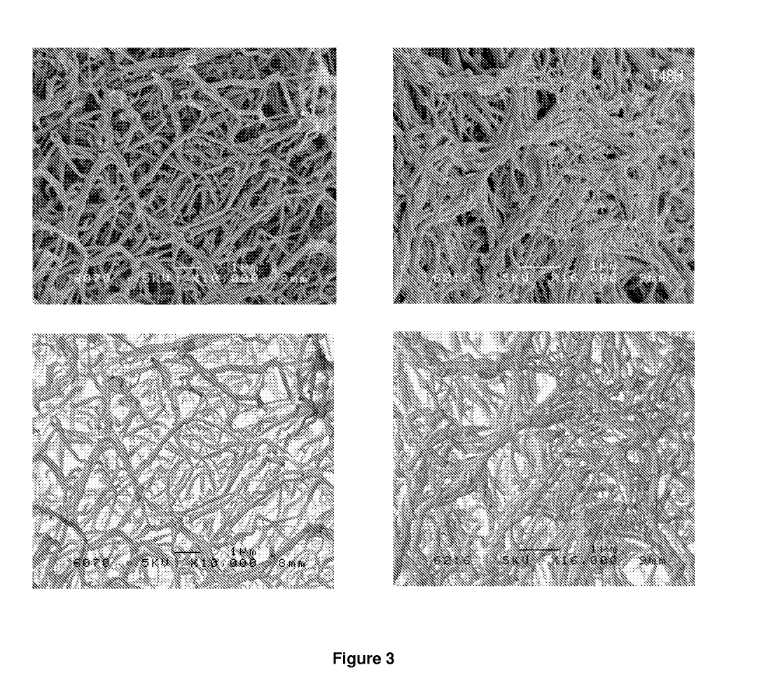

The observations by electron microscopy and photon microscopy show, as represented in FIG. 3, that the tensing agents tested induce reorganization of the collagen fibrils in the dermis after 48 h of application, and also a lengthening and an increase in the number of the fibroblasts.

The collagen fibrils of the extracellular matrix undergo association in fibrillar structure so as to form networks of greater density. This new organization may be linked to the synthesis of proteoglycans, such as decorin or else lumican, which are known to undergo association with a number of collagen molecules and thus to regroup them in a well-ordered network.

The mechanical stresses induced by the tensing agents have therefore stimulated the fibroblasts, leading to a reorganization of their cytoskeleton, and have done so from 48 h after application, which are therefore very short kinetics.

1-37. (canceled)

38. A method for increasing skin thickness, improving skin density, improving skin elasticity, improving skin firmness, improving skin tonicity, promoting skin regeneration, promoting skin renewal, promoting extracellular matrix regeneration, promoting extracellular matrix reorganization, or regulating the expression of genes involved in skin homeostasis via transmission of mechanical tensions involving activation of mechanoreceptors, wherein said method comprises applying a composition to skin, wherein said composition comprises a physiologically acceptable medium and at least one mosaic-effect tensing agent.

39. The method according to claim 38, wherein said method is a method for increasing skin thickness.

40. The method according to claim 38, wherein said method is a method for improving skin density.

41. The method according to claim 38, wherein said method is a method for improving skin elasticity.

42. The method according to claim 38, wherein said method is a method for improving skin firmness.

43. The method according to claim 38, wherein said method is a method for improving skin tonicity.

44. The method according to claim 38, wherein said method is a method for promoting skin regeneration.

45. The method according to claim 38, wherein said method is a method for promoting skin renewal.

46. The method according to claim 38, wherein said method is a method for promoting extracellular matrix regeneration.

47. The method according to claim 38, wherein said method is a method for promoting extracellular matrix reorganization.

48. The method according to claim 38, wherein said method is a method for regulating the expression of genes involved in skin homeostasis via transmission of mechanical tensions involving activation of mechanoreceptors.

49. The method according to claim 38, wherein said method comprises applying the composition to the skin at least twice per week.

50. The method according to claim 38, wherein said method comprises applying the composition to the skin at least once per day.

51. The method according to claim 38, wherein the at least one mosaic-effect tensing agent is selected from the group consisting of one or more mineral tensing agents, one or more tensing polymers and mixtures thereof.

52. The method according to claim 38, wherein the at least one mosaic-effect tensing agent is one or more mineral tensing agents selected from the group consisting of mixed silicates, colloidal particles of inorganic fillers and mixtures thereof.

53. The method according to claim 38, wherein the at least one mosaic-effect tensing agent is one or more synthetic tensing polymers.

54. The method according to claim 38, wherein the at least one mosaic-effect tensing agent is one or more tensing polymers of an interpenetrating polymer network type.

55. The method according to claim 38, wherein the at least one mosaic-effect tensing agent is one or more tensing polymers of an interpenetrating polymer network type comprising a polyurethane polymer and an acrylic polymer.

56. The method according to claim 38, wherein the at least one mosaic-effect tensing agent is one or more tensing polymers comprising: 70-90 wt. % of at least one aryl and/or alkyl acrylate, and/or at least one aryl and/or alkyl methacrylate, and/or styrene; and 10-30 wt. % of (meth)acrylic acid.

57. The method according to claim 38, wherein the at least one mosaic-effect tensing agent is present in an effective amount of 1-30 wt. % based on the total weight of the composition.
